(19) United States
(12) Patent Application Publication
(10) Pub. No.: US 2009/0326616 A1
(43) Pub. Date: Dec. 31, 2009

(54) SYSTEM AND METHOD FOR INFLUENCING A PHOTOBIOLOGICAL STATE

(75) Inventors: Ronaldus Maria Aarts, Eindhoven (NL); Lucas Josef Maria Schlangen, Eindhoven (NL)

Correspondence Address: Philips Intellectual Property and Standards, P.O. Box 3001, Briarcliff Manor, NY, 10510-8001, US

(73) Assignee: KONINKLIJKE PHILIPS ELECTRONICS N.V., Eindhoven (NL)

(21) Appl. No.: 12/373,914

(22) PCT Filed: Jul. 25, 2007
(86) PCT No.: PCT/IB07/52958

§ 371 Date: Jan. 15, 2009

(30) Foreign Application Priority Data
Aug. 7, 2006 (EP) 06118510.4

(51) Int. Cl. A61N 5/06

(52) U.S. Cl. 607/88

(57) ABSTRACT

The invention relates to a system (10) and method for influencing a photobiological state in a vertebrate (5). The system comprises a light source (30, 32) for emitting light which influences the photobiological state, a sensor (20, 22) arranged to sense a first biophysical parameter (P1), and a control circuit (12) for controlling the light source (30, 32) so as to generate a predetermined photobiological state. The biophysical parameter represents a biological state of the vertebrate (5). The control circuit (12) receives a feedback signal (S1, S2) from the sensor (20, 22) and subsequently sends a control signal (16, 17, 18, S3) to the light source (30, 32) for controlling the light source (30, 32). The control signal is generated by combining a second parameter with the first biophysical parameter. The second parameter is a second biophysical parameter or an interaction parameter characterizing an interaction of the vertebrate with a device. The second parameter represents a further biological state of the vertebrate. The second biophysical parameter is sensed, for example, at a different time and/or is a different biophysical parameter as compared to the first biophysical parameter.

### FIELD OF THE INVENTION

The invention relates to a system for influencing a photobiological state in a vertebrate. The invention further relates to a lighting device, a backlighting device, a display device and a method comprising the system.

### BACKGROUND OF THE INVENTION

It is known that the circadian rhythm in humans controls important processes such as the daily cycle of waking and sleeping. This biological clock aligns its timing to the external environment through, for example, exposure to light via the hormone melatonin, which is associated with sleep. The synthesis of melatonin is reduced when light hits the retina of the eye. However, it is sometimes required that the circadian rhythm is influenced, such as temporarily extending the period of waking, for example, when driving a car.

Patent application DE 10232797A1 discloses a system for increasing a driver's vigilance. The system comprises a sensor for sensing a parameter representing the level of attention of the driver, and comprises a light source which emits electromagnetic radiation triggering receptors in the human eye that are responsible for a human's circadian rhythm. The triggering of the receptors suppresses the production of melatonin and increases the driver's vigilance.

The known system has the drawback that it may alter the circadian rhythm or phase of the driver, which results in a reduced well-being.

### OBJECT AND SUMMARY OF THE INVENTION

It is an object of the invention to improve the user's well-being.

In accordance with a first aspect of the invention, this object is achieved with a system for influencing a photobiological state of a vertebrate, the system comprising:

a light source for emitting light influencing the photobiological state of the vertebrate,

a sensor arranged to sense a first biophysical parameter representing a biological state of the vertebrate and generate a feedback signal representing the first biophysical parameter, and

a control circuit arranged to receive the feedback signal from the sensor and generate a control signal controlling the light source for influencing the photobiological state of the vertebrate so as to generate a predetermined photobiological state of the vertebrate,

the control signal being generated by combining a second parameter with the first biophysical parameter, the second parameter being a second biophysical parameter or an interaction parameter characterizing an interaction of the vertebrate with a device, the second parameter representing a further biological state of the vertebrate.

A photobiological state of a vertebrate is a state which is influenced by light, such as alertness, sleep, depression, circadian rhythm, and concentration of the hormones cortisol and melatonin.

The effect of the measures according to the invention is that a control signal, which is based on both the first biophysical parameter and the second parameter, allows more accurate and subtle manipulation of the photobiological state, which improves the well-being of the vertebrate. In the known system, the driver's vigilance is increased as soon as a reduction of his level of attention is sensed. This may result in over-stimulation of the driver resulting, especially during long drives, in a change of the driver's circadian rhythm causing a feeling of jet-lag or depression. The use of the second parameter enables the system to take account of, for example, a previously measured biophysical parameter, or allows taking account of, for example, measurements of a different biophysical parameter related to the first biophysical parameter. Using the previously measured biophysical parameter or the different biophysical parameter allows, for example, detection of a trend in the biophysical parameters which may already indicate that the photobiological state of the vertebrate which must be influenced is changing. When the observed change in the biophysical parameter progresses in a different direction as compared to the required predetermined photobiological state, a different control signal may be necessary as compared to the situation in which the change in the biophysical parameter already progresses in the direction of the required predetermined photobiological state. The synergy between the first biophysical parameter and the second parameter enables the system to determine, for example, exactly at what stage in the circadian rhythm the user is, such that a prediction may be possible of what the user's condition will be when the photobiological state is not influenced, and what is required to obtain the appropriate photobiological state. This may prevent over-stimulation of the vertebrate and increases its well-being.

Alternatively, the second parameter may be an interaction parameter characterizing the interaction of the vertebrate with a device. For example, the keystroke rate at which a person is working on a computer, or the person's steering behavior when driving a car may be a representation of his biological state. Also this interaction parameter enables the system to determine, for example, at what stage in the circadian rhythm the user currently is and what actions are required to obtain the predetermined photobiological state.

The system has the further advantage that it allows a gradual change of the circadian rhythm of the vertebrate, for example, after crossing time zones, or, for example, when adapting to or from night shifts. As the use of the second parameter allows detection of a trend in the biophysical parameter, this trend may be altered to bring the vertebrate back to the circadian rhythm or phase of his choice. Instead of forcing the new circadian rhythm on the vertebrate, a gradual alteration of the trend of the biophysical parameter within the current circadian rhythm enables the current circadian rhythm or phase to be gradually changed into the circadian rhythm of choice while maintaining a relatively high level of well-being for the vertebrate.

In an embodiment of the system, the sensor senses the first biophysical parameter on or in the vertebrate's body. This embodiment has the advantage that the sensing on the vertebrate's body allows a relatively accurate measurement which enables a control signal to more precisely control the photobiological state. This allows control of an amount and a direction of the variation of the photobiological state and prevention of an abrupt change in the photobiological state, thus increasing the vertebrate's well-being.

In an embodiment of the system, the first biophysical parameter and the second parameter are used to determine a phase in a circadian rhythm of the vertebrate. The inventor has realized that the circadian rhythm can be described by using a biophysical parameter which varies in known fashion during the 24-hour cycle of the circadian rhythm. This characteristic variation of the biophysical parameter results in a graph having a characteristic shape. When using, for example, two biophysical parameters measured successively, the known shape of the biophysical parameter may be fitted, which results in a relative accurate determination of the phase in the circadian rhythm of the vertebrate. When using two different biophysical parameters, with the first biophysical parameter having a first characteristic variation in time and the second having a second characteristic variation in time, the combination of the first and second biophysical parameters can be used to relatively accurately determine the phase of the vertebrate in the circadian rhythm. Typically, the time variations of the first and second biophysical parameters are linked.

In an embodiment of the system, the second biophysical parameter is sensed shifted in time with respect to the first biophysical parameter. This embodiment has the advantage that a single sensor is sufficient to accurately determine the circadian rhythm of the vertebrate and allows influencing of the circadian rhythm while maintaining a feeling of well-being in the vertebrate. Furthermore, when the first and second biophysical parameters are identical, this embodiment allows detection of a trend in the change of the first and second biophysical parameters. A comparison of the observed change between the first and second biophysical parameters with the expected change provides additional information about the well-being, state or phase of the circadian rhythm of the vertebrate, which may be used, for example, when altering the photobiological state.

In an embodiment of the system, the second parameter is sensed by a further sensor. When the first biophysical parameter and the second parameter are identical, the further sensor is substantially identical to the sensor. However, the further sensor may also be different as compared to the sensor and may, for example, sense a different physical parameter.

In an embodiment of the system, the sensor and the further sensor are arranged to sense conditions on or in different locations of the vertebrate's body. This embodiment has the advantage that the measurement on different parts of the body provides additional information which can be used to influence the photobiological state. For example, in a preferred embodiment of the invention, the sensor and the further sensor are temperature sensors, wherein the sensor senses conditions at a distal (for example, a hand or a foot) of the vertebrate's body and the further sensor senses conditions at a proximal (for example, stomach or thigh) of the body. A temperature difference between distal and proximal may be used as an indication of an onset of sleep.

In an embodiment of the system, the first biophysical parameter and the second parameter are different. Different biophysical parameters are typically linked to one circadian rhythm of the vertebrate and as such typically linked to each other, behaving coherently. The coherent behavior of the different biophysical parameters may be used, for example, to relatively accurately determine the phase in the circadian rhythm of the vertebrate. Alternatively, the registration of an unexpected and incoherent behavior between the first and the second biophysical parameter may be an indication that the vertebrate is not fit or even ill and may trigger an alarm.

In an embodiment of the system, the first and/or the second biophysical parameter are selected from a group comprising skin temperature, body temperature, breathing depth and frequency, electro-encephalogram, electro-oculogram, heart beat, heart beat rate variability and inter heart beat interval, skin conductance, melatonin concentration, cortisol concentration, and body movement, wherein the interaction parameter is selected from a group comprising keystrokes on a computer, steering in a car, and operating a gas pedal in a car. The electro-encephalogram (further also referred to as EEG) is an indication of the vertebrate's brain activity. The electro-oculogram (further also referred to as EOG) is an indication of the vertebrate's eye movement which is an indication of his alertness.

In an embodiment of the system, the control signal controls color, brightness and/or composition of the light emitted by the light source. It is known that especially blue light having a central wavelength of approximately 460 nm (further also referred to as melatonin suppressing blue light) suppresses the production of melatonin and as such influences the circadian rhythm of the vertebrate. The control signal, for example, controls the amount of emission of melatonin suppressing blue light or, for example, replaces the melatonin suppressing blue light by blue light having a different central wavelength and thus having a reduced suppression of the production of melatonin.

In an embodiment of the system, the light source comprises a plurality of light-emitting elements. The plurality of light-emitting elements is, for example, a plurality of light-emitting diodes (further also referred to as LED), or a plurality of incandescent lamps, or a plurality of low-pressure gas discharge lamps wherein the different lamps comprise different luminescent materials. The different light-emitting elements are preferably dimmable, such that the individual contributions of each light-emitting element can be regulated.

In an embodiment of the system, the feedback signal and/or the control signal are wireless signals. The use of a wireless connection enables a sensor which is applied in the vertebrate's body, such as directly under his skin, or in a capsule which can be swallowed by the vertebrate. The sensor and controller may be part of a body area network (further also referred to as BAN).

In an embodiment of the system, the control signal is generated by combining a third parameter with the first biophysical parameter and the second parameter, the third parameter being selected from a group comprising local time, local date, recent change of time zone, current ambient environmental conditions and recent changes in ambient environmental conditions. Current ambient environmental conditions include, for example, ambient light conditions, ambient temperature conditions, ambient humidity conditions, current climate and weather. The third parameter provides an indication of, for example, the difference between the circadian rhythm of the vertebrate and the circadian rhythm to which the vertebrate wants to adapt.

In an embodiment of the system, influencing of the photobiological state comprises increasing alertness, stabilizing a circadian rhythm, deviating from a circadian rhythm, changing from one circadian rhythm to a further circadian rhythm, improving physiological performance, or controlling the effectivity of the digestive system prior to or during a meal. The increase of alertness may result in, for example, increased safety or optimal performance during, for example, studying for an exam. Changing one circadian rhythm by a further circadian rhythm may be beneficial, for example, when adapting to night shift periods or travelling across time zones. Improved physiological performance includes, for example, improved performance in sporting events.

The invention also relates to a lighting device, a backlighting system for illuminating a display of a display device, and a display device. The lighting device may be used, for example, in an office for illumination during office hours and allows a smooth synchronization of the circadian rhythm of a person working in the office with the current day and night cycle outside, or for improving synchronization with a working/training/(sports)match schedule. Alternatively, the lighting device may be, for example, a desk lamp which may be used for increasing alertness during studying. The backlighting system may be used, for example, for illuminating a liquid crystal display of, for example, a monitor or an LCD television. The backlighting system in a monitor, which is used in an office environment, may be arranged to either allow smooth synchronization of the circadian rhythm of the person working with the monitor with the current day and night cycle outside. Alternatively, the backlighting system may increase alertness, for example, to temporarily optimally function during office hours. The backlighting system in an LCD television may be arranged to prevent any increase in alertness and optimize, for example, sleep at night.

### BRIEF DESCRIPTION OF THE DRAWINGS

These and other aspects of the invention are apparent from and will be elucidated with reference to the embodiments described hereinafter.

In the drawings:

FIG. 1 is a schematic representation of the system according to the invention,

FIG. 2 shows a circadian rhythm of the body temperature of a human,

FIG. 3 shows a circadian rhythm of the hormones melatonin and cortisol, and

FIGS. 4A and 4B are schematic representations of an illumination system and a backlighting system according to the invention, respectively.

The Figures are purely diagrammatic and not drawn to scale. Particularly for clarity, some dimensions are exaggerated strongly. Similar components in the Figures are denoted by the same reference numerals as much as possible.

### DESCRIPTION OF EMBODIMENTS

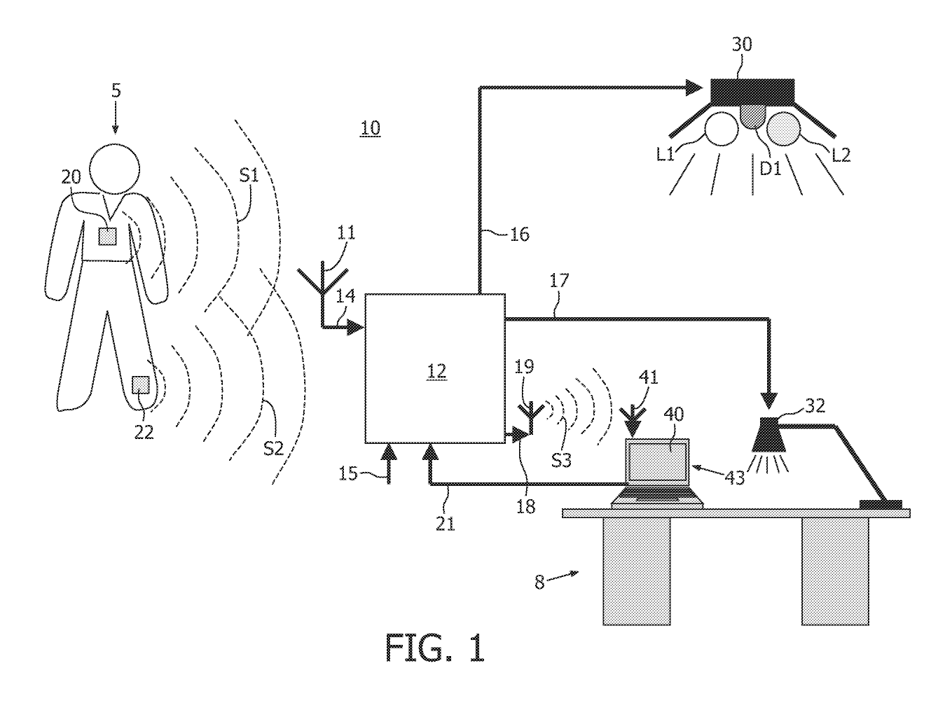

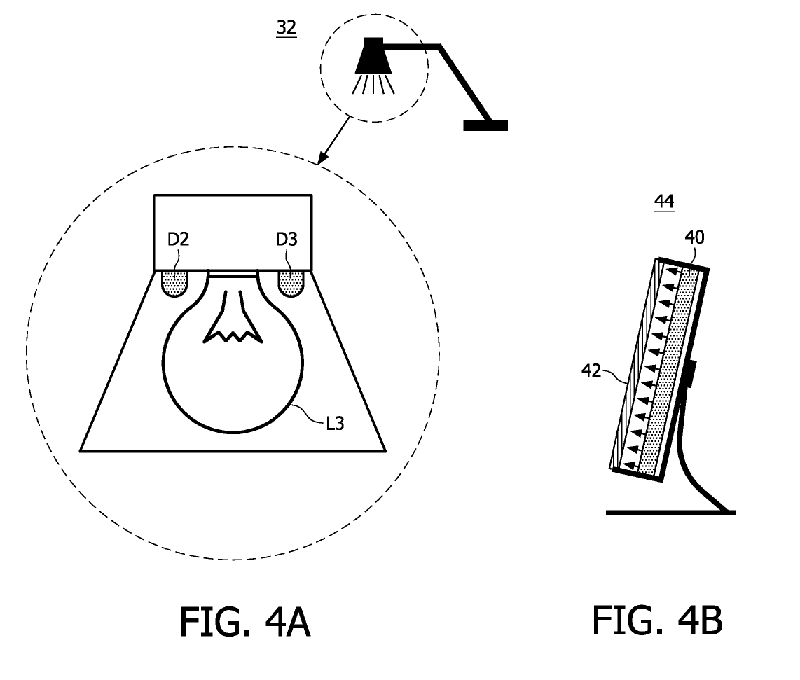

FIG. 1 is a schematic representation of the system 10 according to the invention. The system comprises a light source 30, 32 for emitting light influencing a photobiological state in a vertebrate 5 which is represented by a human 5 in FIG. 1. Examples of light sources shown in FIG. 1 are a luminaire 30, typically a light fitting in an office which can be applied to the ceiling, a desk lamp 32 for illuminating an area on an office desk, and a backlighting system 40 for illuminating a display 42 (see FIG. 4B) of a liquid crystal display device 44 (see FIG. 4B). The light sources 30, 32 are arranged to emit light which influences a photobiological state, such as the production of melatonin. Melatonin is produced during the night. When light, especially blue light, hits the retina of an eye of the human 5 while melatonin is being produced, the production of melatonin is suppressed. Light exposure during the day can enhance the nocturnal melatonin peak which is associated with better sleep. Influencing the photobiological state comprises shifting a phase in the circadian rhythm of the human. This can be used, for example, when the human wants to get up early, or when adapting to or from night shifts. Other changes of the photobiological state are, for example, inducing alertness, reducing depression, and improving sleep quality. The system 10 further comprises a sensor 20, 22 for sensing a first biophysical parameter. Examples of biophysical parameters are skin or body temperature of the human 5, an electro-encephalogram (further also referred to as EEG) which is an indication of the brain activity, an electro-oculogram (further also referred to as EOG) which is an indication of the eye movement, which indicates a level of alertness, a heart beat rate, skin conductance, or body movement. Each biophysical parameter represents a biological state of the vertebrate 5, which may be an indication of, for example, the level of alertness or a phase of the circadian rhythm of the human 5, which contains information about the position or position change within the circadian rhythm. The sensor 20, 22 is able to sense the first biophysical parameter and to transmit a feedback signal S1, S2 to a control circuit 12. The feedback signal S1, S2 is a representation of the biophysical parameter sensed by the sensor 20, 22. In FIG. 1, the feedback signal S1, S2 is indicated as waves progressing through the air surrounding the human 5, indicating that the feedback signal is transmitted from the sensor 20, 22 to the control circuit 12 via a wireless connection such as a body area network. Alternatively, the sensor 20, 22 may be connected to the control circuit 12 via wires.

The system 10 also comprises a control circuit 12. The control circuit 12 receives the feedback signal S1, S2 from the sensor 20, 22 and transmits a control signal 16, 17, 18, S3 to the light source 30, 32 for controlling the light source 30, 32 so as to obtain a predetermined photobiological state. The predetermined photobiological state may be an increased alertness of the human 5, or a stabilization of his circadian rhythm, for example, after a disturbance caused, for example, by prolonged exposure to blue light, or a deviation in the circadian rhythm, for example, extending the phase of alertness required to do additional studies before an exam, or a change from one circadian rhythm to a further circadian rhythm, for example, when crossing time zones during traveling. The control circuit 12 comprises a second parameter which represents a further biological state of the human 5. The second parameter may be a second biophysical parameter P2 or an interaction parameter characterizing the interaction of the vertebrate with a device 43, for example, a computer 43 or a car (not shown). The control signal 16, 17, 18, S3 is determined by combining the first with the second biophysical parameter.

The second biophysical parameter may be, for example, the same biophysical parameter as the first, either sensed time shifted with respect to the first biophysical parameter or sensed at a different location on the body of the human 5. The interaction parameter may be, for example, the keystroke rate at which a person is working on a computer 43, or his steering behavior when driving a car (not shown). The combination of the first biophysical parameter with the second parameter may be, for example, a difference between the first and the second biophysical parameter, for example, a temperature difference between a body temperature at one location on the physical structure of the human 5 sensed at different times within his circadian rhythm and, for example, a body temperature at different locations on the body of the human 5. Alternatively, the second parameter may be, for example, a different biophysical parameter as compared to the first biophysical parameter. For example, the first biophysical parameter may be a body temperature and the second biophysical parameter may be a heart beat rate, or a melatonin concentration and a cortisol concentration, respectively. Typically, each of these different biophysical parameters has a characteristic variation during the circadian rhythm of the human 5. When the first and the second biophysical parameter represent different biophysical parameters, the combination may also result in, for example, fitting two curves through the sensed values of the first and second biophysical parameters, each curve representing the variation of one of the first or the second biophysical parameter during the circadian rhythm of the human 5. In this way, a relatively good estimate of the phase in the circadian rhythm of the specific human 5 can be determined. This estimate of the phase of the circadian rhythm may be used to determine what control signal is necessary to obtain the required predetermined photobiological state while substantially maintaining a feeling of well-being by the human 5.

In FIG. 1, the control circuit 12 has a first input terminal, which is an antenna 11 for receiving the wireless feedback signal S1, S2, and a second input terminal 21 for receiving the interaction parameter, which in FIG. 1 represents the interaction of the human 5 with a computer 43. The control circuit 12 has a further input terminal 15 for receiving a third parameter, such as local time, a recent change of time zone, current ambient environmental conditions surrounding the human 5 and recent changes in these conditions. Current ambient environmental conditions include, for example, conditions of ambient light, temperature, humidity, current climate and weather. This third parameter may be used to generate the control signal 16, 17, 18, S3, for example, to determine a difference between the circadian rhythm of the human 5 and the local day and night rhythm to which he wants to adapt. The control circuit 12 further has a first output terminal for sending a control signal 16 to the luminaire 30, a second output terminal for sending a control signal 17 to the desk lamp 32, and a third output terminal connected to a second antenna 19 for sending a control signal 18, S3 to an antenna 41 connected to the backlighting system 40 of the display device 44.

The light source 30, 32 of the system 10 according to the invention is arranged to emit light influencing the photobiological state. In FIG. 1, this is indicated by the luminaire 30 comprising a first light-emitting element L1, a second light-emitting element L2 and a third light-emitting element D1. The first and the second light-emitting element L1, L2 are, for example, low-pressure gas discharge lamps L1, L2 which are arranged to emit light having a predetermined color. The color emitted by the low-pressure gas discharge lamps L1, L2 is generally determined by the phosphor or mixture of phosphors used in these lamps and can generally not be altered (other than exchanging the low-pressure gas discharge lamp with a different lamp having a different mixture of phosphors). The low-pressure gas discharge lamps L1, L2 may be dimmable, thus altering their intensity contribution. The luminaire 30 shown in FIG. 1 further comprises a third light-emitting element D1, for example, a light-emitting diode D1 (further also referred to as LED). In the embodiment shown in FIG. 1, the first, second and third light-emitting elements L1, L2, D1 are dimmable elements, all of which emit light of a different color. This configuration constitutes a light source 30 which can emit light of a wide range of different colors and intensities.

In FIG. 1, there are several sensors 20, 22 applied on the body of the human 5. These sensors 20, 22 may sense, for example, the same biophysical parameter at different locations on the body of the human 5. For example, a first sensor 20 senses a body temperature at a proximal of the body of the human 5, for example, the body temperature at his thigh or stomach. A second sensor 22 senses, for example, a body temperature at a distal of the body of the human 5, for example, at a hand or a foot. The difference between the body temperature at the distal as compared to the body temperature at the proximal allows an estimate of the current phase of the circadian rhythm of a particular human 5. Alternatively, the first sensor 20 may sense, for example, the first biophysical parameter, for example, a concentration of melatonin, and the second sensor 22 may sense, for example, the second biophysical parameter, for example, a concentration of cortisol. The first and the second sensor 20, 22 may be applied to the skin of the human 5, or somewhere underneath his skin. For example, the sensor 20 may be applied in a capsule and swallowed by the human 5 to accurately sense the body temperature in his stomach. Alternative methods of applying the sensors to the body of the human 5 are well known to the skilled person.

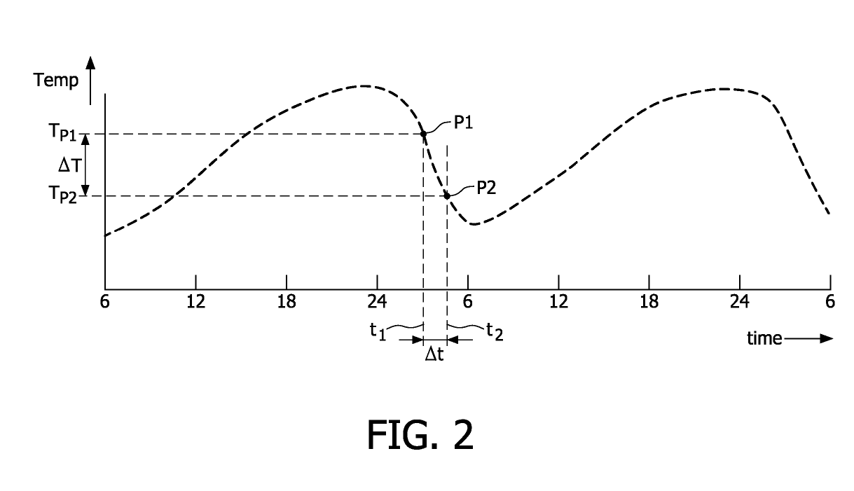

FIG. 2 shows a circadian rhythm of the body temperature of the human 5. In the graph shown in FIG. 2, time is plotted on the horizontal axis and temperature is plotted on the vertical axis. The body temperature of a human is at its lowest in the morning just before he wakes up. During the day, the body temperature gradually increases and arrives at a peak during the night when the human 5 normally sleeps. In the second part of the night, normally during the sleep phase of his circadian rhythm, the temperature drops again. As can clearly be seen from FIG. 2, the body temperature variation of the human 5 has a characteristic shape during the 24-hour cycle of the circadian rhythm. When a first body temperature T_{P1}, i.e. a first biophysical parameter P1, is measured at a time t₁ and a second body temperature T_{P2}, i.e. the second biophysical parameter P2, is measured at t₂, the temperature difference ΔT between the two biophysical parameters P1, P2 allows determination of an estimate of the current phase of the circadian rhythm of a particular human 5. When the current phase of the circadian rhythm is known, the control circuit 12 (see FIG. 1) can determine which change of the photobiological state is required to obtain the predetermined photobiological state. Furthermore, a comparison of the time difference Δt with the sensed temperature difference ΔT enables the control circuit to determine a trend in the biophysical parameter P1, P2 which can be used to better control the influencing of the photobiological state so as to obtain the predetermined photobiological state.

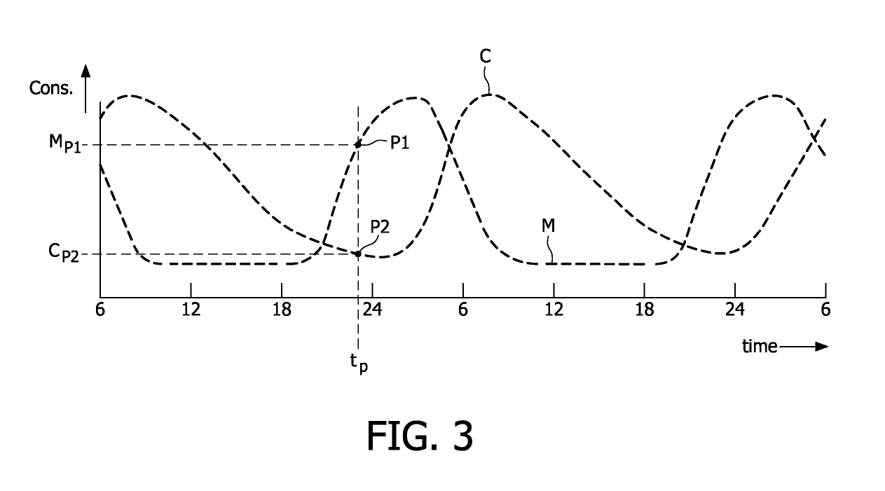

FIG. 3 shows a circadian rhythm of the hormones melatonin M and cortisol C. In the graph shown in FIG. 3, time is plotted on the horizontal axis and a concentration of the hormone melatonin M or cortisol C is plotted on the vertical axis. Sensing melatonin M, i.e. the first biophysical parameter P1, a first concentration M_{P1}, i.e. the concentration of melatonin M is determined by using, for example, the first sensor 20. Sensing cortisol C, i.e. the second biophysical parameter P2, a second concentration C_{P2}, i.e. the concentration of cortisol C is determined by using, for example, the second sensor 22. As can be seen in FIG. 3, the variation of concentration of both melatonin M and cortisol C have a characteristic shape during the 24-hour cycle of the circadian rhythm. When, at a certain time t_{P}, the concentrations of both melatonin M and cortisol C are sensed, the sensed concentrations M_{P1} and C_{P2} can be used to determine an estimate of the current phase of the circadian rhythm of the particular human 5, which estimate can be subsequently used to determine which change of the photobiological state is required to obtain the predetermined photobiological state. In FIG. 3, the concentrations of both melatonin M and cortisol C are sensed at the same time. Alternatively, the concentrations of melatonin M and cortisol C may be sensed at different times during the circadian rhythm, for example, at times when both the melatonin concentration M_{P1} and the cortisol concentration C_{P2} are expected to have a maximum value. The concentrations of both melatonin M and cortisol C may also be sensed by using a single sensor which is able to sense both biophysical parameters P1, P2.

FIGS. 4A and 4B are schematic representations of an illumination system 32 and a backlighting system 40 according to the invention, respectively. The illumination system 32 of FIG. 4A is the desk lamp 32 as already shown in FIG. 1. In FIG. 4A, the light-emitting part of the desk lamp 32 is shown in more detail, with a fourth light-emitting element L3, typically a tungsten lamp L3, a fifth light-emitting element D2, typically a LED, and a sixth light-emitting element D3, typically a further LED. The tungsten lamp L3 emits, for example, substantially white light of a predetermined color for clear illumination of the surface of the desk 8 (see FIG. 1). The LEDs D2, D3 emit, for example, light of a specific color, for example, blue, to influence the photobiological state. Especially the use of blue light having a predominant wavelength between 440 and 495 nanometers, and more specifically between 460 and 475 nanometers, results in a strong suppression of the nocturnal secretion of the hormone melatonin when illuminating the retina of a human 5. Varying the contribution of blue light emitted by the LEDs D2, D3 allows a change in the circadian rhythm of the human 5.

In an embodiment of the desk lamp 32 according to the invention, the combined emission of the tungsten lamp L3 and the first LED D2 provides a first color emitted by the desk lamp 32, which changes the photobiological state of the human to, for example, an increased alertness. The combined emission of the tungsten lamp L3 and the second LED D3 provides, for example, a second color emitted by the desk lamp 32, which changes the photobiological state of the human differently to, for example, a decreased alertness.

An embodiment of the desk lamp 32 may also comprise the control circuit 12 (not shown in FIG. 4A) for receiving the feedback signal S1, S2 (see FIG. 1) from the sensor 20, 22 (see FIG. 1) and for sending a control signal (not shown) to the light-emitting elements L3, D2, D3 of the desk lamp 32 for controlling these elements so as to obtain the predetermined photobiological state.

FIG. 4B is a schematic representation of a display device 44 having a display 42 and a backlighting system 40 according to the invention. The display 42 is typically a non-emissive display, such as an array of liquid crystal cells which, by varying the transmission of cells in the array, is able to create an image on the display 42. The backlighting system 40 may comprise a plurality of lighting devices, such as a plurality of low-pressure gas discharge lamps (not shown), which emit light of a different color and can be dimmed individually. When a particular intensity combination of the different low-pressure gas discharge lamps is chosen, the emitted light comprises a required color and intensity to influence the photobiological state. Alternatively, the backlighting system 40 may comprise a plurality of light-emitting diodes (not shown) or lasers (not shown) emitting a specific color. By changing the intensity of the LEDs or lasers, or by changing the number of LEDs or lasers which contribute to the light emitted by the display 42, a required color and intensity of the emitted light can be determined to influence the photobiological state. The backlighting system 40 may comprise, for example, a waveguide (not shown) for mixing the different color contributions of the different light-emitting elements so as to obtain a uniform light distribution across the display 42.

In an embodiment of the display device 44, the display device 44 or the backlighting system 40 may comprise the control circuit 12 for receiving the feedback signal S1, S2 (see FIG. 1) from the sensor 20, 22 (see FIG. 1) and for sending a control signal (not shown) to the light-emitting elements of the backlighting system 40 for controlling these elements so as to obtain the predetermined photobiological state.

Alternatively, the display device 44 may be a cathode ray tube display device.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. Use of the article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A system for influencing a photobiological state of a vertebrate, the system comprising:
a light source for emitting light influencing the photobiological state of the vertebrate,
a first sensor arranged to sense a first biophysical parameter (P1) representing a biological state of the vertebrate and generate a feedback signal (S1) representing the first biophysical parameter (P1), and
a control circuit arranged to receive the feedback signal (S1) from the sensor and generate a control signal controlling the light source for influencing the photobiological state of the vertebrate so as to generate a predetermined photobiological state of said vertebrate,
the control signal being generated by combining a second parameter with the first biophysical parameter (P1), the second parameter being a second biophysical parameter (P2) being sensed shifted in time (Δt) with respect to the first biophysical parameter (P1) or an interaction parameter characterizing an interaction of the vertebrate with a device, the second parameter representing a further biological state of the vertebrate.

2. A system as claimed in claim 1, wherein the sensor senses the first biophysical parameter (P1) on or in the body of a vertebrate.

3. A system as claimed in claim 1, wherein the first biophysical parameter (P1) and the second parameter (P2) are used to determine a phase in a circadian rhythm of the vertebrate.

4. (canceled)

5. A system as claimed in claim 1, wherein the second parameter is sensed by a second sensor (22).

6. A system as claimed in claim 5, wherein the first sensor and the second sensor are arranged to sense conditions on or in different parts of the body of the vertebrate.

7. A system as claimed in claim 5, wherein the first biophysical parameter (P1) and the second parameter (P2) are different biophysical parameters.

8. A system as claimed in claim 1, wherein the first and/or the second biophysical parameters (P1, P2) are selected from a group consisting of: skin temperature, body temperature, breathing depth and frequency, electro-encephalogram, electro-oculogram, heart beat, heart beat rate variability and inter heart beat interval, skin conductance, melatonin concentration, cortisol concentration, and body movement, and wherein the interaction parameter is selected from a group consisting of: keystrokes on a computer, steering a car, and operating a gas pedal in a car.

9. A system as claimed in claim 1, wherein the control signal controls color, brightness and/or composition of the light emitted by the light source.

10. A system as claimed in claim 1, wherein the light source emits light having a wavelength which is shorter than 500 nm.

11. A system as claimed in claim 1, wherein the light source comprises a plurality of light-emitting elements (D1, D2, L).

12. A system as claimed in claim 1, wherein the feedback signal and/or the control signal (S3) are a wireless signal.

13. A system as claimed in claim 1, wherein the control signal is generated by combining a third parameter with the first biophysical parameter (P1) and the second parameter, the third parameter being selected from a group comprising local time, local date, recent change of time zone, current ambient environmental conditions and recent changes in ambient environmental conditions.

14. A system as claimed in claim 1, wherein the influencing of the photobiological state comprises increasing alertness, stabilizing a circadian rhythm, deviating from a circadian rhythm, changing from one circadian rhythm to a further circadian rhythm, improving physiological performance, or controlling the effectivity of the digestive system prior to or during a meal.

15-17. (canceled)

18. A method of influencing a photobiological state of a vertebrate, using a light source for emitting light influencing the photobiological state of the vertebrate, the method comprising the steps of:
sensing a first biophysical parameter (P1) representing a first biological state of the vertebrate, and
generating a control signal controlling the light source for influencing the photobiological state of the vertebrate so as to generate a predetermined photobiological state of said vertebrate, the control signal being generated by combining a second parameter with the first biophysical parameter (P1), the second parameter being a second biophysical parameter (P2) or an interaction parameter characterizing an interaction of the vertebrate with a device, the second parameter representing a further biological state of the vertebrate.
